(19) United States
(12) Patent Application Publication
(10) Pub. No.: US 2022/0257645 A1
(43) Pub. Date: Aug. 18, 2022

(54) PHARMACEUTICAL COMPOSITION FOR PREVENTING OR TREATING METABOLIC BONE DISEASES, CONTAINING BENTONITE AS ACTIVE INGREDIENT

(71) Applicant: Korea Institute Of Geoscience And Mineral Resources, Daejeon (KR)

(72) Inventors: Sung Man SEO, Pohang-si (KR); Il-Mo KANG, Seoul (KR); Ki Min ROH, Daejeon (KR); Jae Hwan KIM, Pohang-si (KR); Dae Young KIM, Chungcheongnam-do (KR); Jeung-Hee AN, Seoul (KR)

(73) Assignee: Korea Institute Of Geoscience And Mineral Resources, Daejeon (KR)

(21) Appl. No.: 17/627,926

(22) PCT Filed: Jul. 16, 2020
(86) PCT No.: PCT/KR2020/009354

§ 371 Date: Jan. 18, 2022

(30) Foreign Application Priority Data
Jul. 18, 2019 (KR) 10-2019-0087226

(51) Int. Cl. A61K 33/06; A61P 19/00; A23L 33/16

(52) U.S. Cl. CPC A61K 33/06; A23L 33/16; A61P 19/00

(57) ABSTRACT

Provided is a pharmaceutical composition for preventing or treating metabolic bone diseases, containing bentonite as an active ingredient. The present disclosure relates to a composition for inhibiting bone resorption of osteoclasts and simultaneously, promoting bone formation of osteoblasts to be usable in the prevention or treatment of metabolic bone diseases such as osteoporosis, osteomalacia, osteopenia and bone atrophy.

### TECHNICAL FIELD

The present disclosure relates to a pharmaceutical composition for preventing or treating metabolic bone diseases, containing bentonite as an active ingredient, and more particularly, to a composition that is usable in the prevention or treatment of metabolic bone diseases such as osteoporosis, osteomalacia, osteopenia, and bone atrophy.

### BACKGROUND ART

Metabolic bone diseases are caused when the activity is unbalanced between osteoblasts, which form bones in the body, and osteoclasts, which plays a role in destroying bones. Osteoclasts are large multinucleated cells that destroy or resorb bone tissues that are unnecessary in the process of bone formation. Matured osteoclasts are multinucleated cells and originate from hematopoietic stem cells. Osteoblasts differentiated from mesenchymal stem cells survive for about 34 months, and form a new bone at a site where activated osteoclasts decompose an old bone. A lot of osteoblasts make a bone matrix, and bone formation is completed as the matrix gradually becomes mineralized. Thereafter, more than about 70% of osteoblasts are killed, and some osteoblasts are differentiated into osteocytes and bone lining cells and survive. Since the amount of bone is maintained by the balance of osteoclasts and osteoblasts, it is important to develop therapeutic agents targeting molecules that play an important role in osteoclasts. In other words, when the activity of osteoclasts that resorb bone increases, diseases such as osteoporosis, in which bone decomposition is promoted, and bones become thin and break easily, are caused, so that factors capable of controlling the activity of osteoclasts have been studied as therapeutic agents for bone diseases.

For example, osteopenia refers to the pre-stage of osteoporosis, and the cause thereof is known to be caused by resorption and formation of excessive osteoclasts. Bone atrophy shown in rheumatism is also associated with resorption of excessive osteoclasts. In fibrous dysplasia, osteoclasts are active. In Paget disease and hypercalcemia, therapeutic agents through the inhibition of functions of osteoclasts have been used. The formation and/or activity of excessive osteoclasts are inhibited to inhibit neoplastic bone destruction, and osteolysis and osteoarthritis are caused by increased resorption of osteoclasts or increased differentiation of osteoclasts.

On the other hand, it is known that cancer cells penetrated into the bone are proliferated in a microenvironment around the bone to stimulate the activity of osteoclasts or osteoblasts, thereby determining whether to progress to osteolytic bone metastasis or osteoblastic bone metastasis. Cancer cells that travel along blood vessels and are settled in the bones secrete osteolytic factors such as parathyroid hormone-related protein (PTHrP), interleukin (IL)-1, IL-6, IL-8 and IL-11. The secreted factors decrease the expression of osteoprotegerin (OPG) and increase the expression of a receptor activator of NF-kB ligand (RANKL) in osteoblasts. Increased RANKL binds with RANK of osteoclast progenitors to muture a large number of osteoclast progenitors, and eventually cause bone destruction by excessive bone resorption.

On the other hand, clay minerals are known to change physicochemical properties through adsorption and release of various ingredients due to a wide active surface area due to the characteristics of particles and structures. Among these clay minerals, bentonite as one of layered silicate minerals is produced by the transformation of volcanic ash, mainly composed of smectite minerals, and composed of the clay of montmorillonite minerals as a main ingredient. Therefore, bentonite mainly refers to impure montmorillonite, and is defined as colloidal hydrous aluminum silicate produced in nature in the Korean Food Additives Codex.

Currently, in Korea, the utilization of these clay minerals as medicinal raw materials is low, and bentonite is abundantly buried in Korea, but contains impure minerals such as quartz, feldspar, mica, iron oxide, and silica hydrate, and has a low montmorillonite content to be mainly used limitedly for molding sand, binder, civil engineering, and the like. However, recent attempts have been made to develop basic materials for medicinal use of mineral resources to convert mineral resources into raw materials for pharmaceuticals, and particularly, bentonite is expected to be used as a high value-added mineral resource in the future and has been in the spotlight.

Conventionally, there has been a composition for treating bone diseases containing bentonite, which is used as a disintegrant added to a pharmaceutical composition to promote decomposition and release the drug, or as an additive to control the turbidity and color of the extract, and bentonite itself was not used as an active ingredient capable of treating bone diseases.

Therefore, the present inventors conducted studies on effects of promotion of differentiation of osteoblasts and differentiation of osteoclasts required for bone formation and fracture recovery of bentonite itself, produced a composition containing bentonite as an active ingredient by inhibiting bone resorption of osteoclasts and simultaneously, promoting bone formation of osteoblasts, and then completed the present disclosure.

### DISCLOSURE

### Technical Problem

An object of the present disclosure is to provide a pharmaceutical composition for preventing or treating metabolic bone diseases, containing bentonite as an active ingredient.

Another object of the present disclosure is to provide a health functional food for preventing or alleviating metabolic bone diseases, containing bentonite as an active ingredient.

Yet another object of the present disclosure is to provide a method for treating metabolic bone diseases using an effect of bentonite itself for treating metabolic bone diseases.

### Technical Solution

An aspect of the present disclosure provides a pharmaceutical composition for preventing or treating metabolic bone diseases, containing bentonite as an active ingredient.

The bentonite may be any one selected from the group consisting of bentonite in which interlayer ions are unsubstituted, bentonite in which interlayer ions are substituted with sodium ions (Na⁺), bentonite in which interlayer ions are substituted with magnesium ions (Mg²⁺), and bentonite in which interlayer ions are substituted with potassium ions (K⁺).

The metabolic bone disease may be osteoporosis, osteomalacia, osteopenia, bone atrophy, fibrous dysplasia, Paget's disease, hypercalcemia, neoplastic destruction of bone, cancer-related bone resorption disease, osteolysis, osteoarthritis, or rheumatoid arthritis.

The bentonite may inhibit bone resorption of osteoclasts and promote bone formation of osteoblasts at the same time.

The bentonite may include 50 to 70 wt % of SiO₂, 10 to 25 wt % of Al₂O₃, 1 to 5 wt % of Fe₂O₃, and 3 to 6 wt % of MgO.

The bentonite may further include at least one selected from the group consisting of 0 to 5 wt % of CaO, 0 to 5 wt % of K₂O, and 0 to 5 wt % of Na₂O.

Another aspect of the present disclosure provides a health functional food for preventing or alleviating metabolic bone diseases, containing bentonite as an active ingredient.

### Advantageous Effects

According to the present disclosure, the pharmaceutical composition or the health functional food containing bentonite as an active ingredient inhibits differentiation and bone resorption of osteoclasts and simultaneously, promotes differentiation and bone formation of osteoblasts to be usable in the prevention, alleviation or treatment of metabolic bone diseases such as osteoporosis, osteomalacia, osteopenia and bone atrophy.

In addition, according to the present disclosure, when bentonite itself is used as an active ingredient for treating bone diseases, there is an advantageous effect of reducing costs compared to using bentonite as an additive and a disintegrant of a conventional composition for treating bone diseases.

### DESCRIPTION OF DRAWINGS

FIG. 1 is a graph of comparing results of MTT assay after treating bentonite samples to MG-63 osteoblasts according to an exemplary embodiment of the present disclosure.

FIG. 2 is a graph of comparing results of alizarin red-S staining after treating bentonite samples to MG-63 osteoblasts according to an exemplary embodiment of the present disclosure.

FIG. 3 is a graph of comparing effects on ALP activity after treating bentonite samples to MG-63 osteoblasts according to an exemplary embodiment of the present disclosure.

FIG. 4 is a graph of comparing results of MTT assay after treating bentonite samples to RAW 264.7 osteoclasts according to an exemplary embodiment of the present disclosure.

FIG. 5 is a graph of comparing effects on TRAP activity in RAW 264.7 cells of bentonite samples according to an exemplary embodiment of the present disclosure.

### BEST MODE FOR THE INVENTION

The present disclosure provides a pharmaceutical composition for preventing or treating metabolic bone diseases, containing bentonite as an active ingredient.

The bentonite may be any one selected from the group consisting of bentonite in which interlayer ions are unsubstituted, bentonite in which interlayer ions are substituted with sodium ions (Na⁺), bentonite in which interlayer ions are substituted with magnesium ions (Mg²), and bentonite in which interlayer ions are substituted with potassium ions (K⁺).

The metabolic bone disease may be osteoporosis, osteomalacia, osteopenia, bone atrophy, fibrous dysplasia, Paget's disease, hypercalcemia, neoplastic destruction of bone, cancer-related bone resorption disease, osteolysis, osteoarthritis, or rheumatoid arthritis.

The bentonite may inhibit bone resorption of osteoclasts and promote bone formation of osteoblasts at the same time.

The bentonite may include 50 to 70 wt % of SiO₂, 10 to 25 wt % of Al₂O₃, 1 to 5 wt % of Fe₂O₃, and 3 to 6 wt % of MgO.

The bentonite may further include at least one selected from the group consisting of 0 to 5 wt % of CaO, 0 to 5 wt % of K₂O, and 0 to 5 wt % of Na₂O.

Further, the present disclosure provides a health functional food for preventing or alleviating metabolic bone diseases, containing bentonite as an active ingredient.

### MODE FOR THE INVENTION

In the following description, only parts required to understand embodiments of the present disclosure will be described, and it should be noted that the description of other parts will be omitted within a range without departing from the gist of the present disclosure.

Terms and words used in the present specification and claims should not be interpreted as being limited to typical or dictionary meanings, but should be interpreted as meanings and concepts which comply with the technical spirit of the present disclosure, based on the principle that the present inventor can appropriately define the concepts of the terms to describe his/her own invention in the best manner. Therefore, the exemplary embodiments described in the present specification and the configurations illustrated in the drawings are merely the most preferred embodiment of the present disclosure and are not intended to represent all of the technical ideas of the present disclosure, and thus, it should be understood that various equivalents and modifications capable of replacing the exemplary embodiments at the time of this application.

Hereinafter, the present disclosure will be described in detail.

The present disclosure provides a pharmaceutical composition for preventing or treating metabolic bone diseases, containing bentonite as an active ingredient.

The term “prevention” used herein refers to all actions that inhibit or delay the onset of metabolic bone diseases by administering a pharmaceutical composition for preventing or treating metabolic bone diseases containing bentonite as an active ingredient according to the present disclosure.

The term “treatment” used herein refers to all actions that alleviate or beneficially change symptoms of metabolic bone diseases by administering a pharmaceutical composition for preventing or treating metabolic bone diseases containing bentonite as an active ingredient according to the present disclosure.

The “metabolic bone diseases”, which are diseases to be alleviated, prevented or treated by the composition of the present disclosure, refer to conditions or diseases caused by excessive formation and/or activity of osteoclasts, and include bone mass loss diseases. The bone mass loss diseases refer to conditions or diseases which exhibit the loss of bone mass accompanied by symptoms such as a decrease in bone density, softening of bone tissue, and the like. Non-limiting examples of the metabolic bone diseases include osteoporosis, osteomalacia, osteopenia, bone atrophy, fibrous dysplasia, Paget's disease, hypercalcemia, neoplastic destruction of bone, cancer-related bone resorption disease, osteolysis, osteoarthritis, rheumatoid arthritis, or the like.

The “osteoporosis” is a condition in which the calcification of bone tissue is reduced, the compact substance of the bone is thinned, and thus the bone marrow cavity is widened, and as the symptom progresses, the bones become weaker, and thus, it is easy to fracture even with a small impact. The bone mass is affected by various factors, such as genetic factors, nutritional intake, a change in hormones, exercise, differences in lifestyle, and the like, and the causes of osteoporosis are known as old age, lack of exercise, low body weight, smoking, low-calcium diets, menopause, oophorectomy, and the like. On the other hand, although there are individual differences, black people have higher bone mass than white people due to a lower bone resorption level, and generally, the bone mass is the highest at 14 to 18 years old, and decreases by about 1% per year in old age. In particular, in the case of women, the bone loss continues to progress after 30 years old, and when reaching menopause, the bone loss progresses rapidly due to hormonal changes. That is, the estrogen concentration rapidly decreases upon reaching menopause, and at this time, like interleukin-7 (IL-7), a large amount of B-lymphocytes are generated to accumulate pre-B cells in the bone marrow, and as a result, the amount of IL-6 is increased to increase the activity of osteoclasts, resulting in a decrease in bone mass.

The “osteomalacia” refers to a symptom in which bones are bent while calcium is not mixed with bones and soft bones are formed when vitamin D is insufficient or there is a kidney disease that excretes a large amount of calcium.

The “osteopenia” refers to a state before osteoporosis, and refers to a state before the bone is perforated as the bone continues to become thinner and lighter.

In the present disclosure, the bentonite is one of smectite-based clay minerals, and includes a montmorillonite mineral unless otherwise specified.

The bentonite is a kind of clay minerals having montmorillonite as a main constituent mineral, and the names of montmorillonite and bentonite are derived from the Montmorillon district, France and the Port Benton district, Wyoming, USA, and montmorillonite is a mineral belonging to a semi-group in mineralogical classification, and a fine aggregate having a crystal structure of Si-tetrahedral layers and Al-octahedral layers of 2:1.

Ca²⁺ and Mg²⁺ ions are the most common types of cations present between the layers of bentonite, and small amounts of Na⁺, H⁺, and K⁺ exist, and a divalent cation has a stronger binding force with a tetrahedral silica layer than a monovalent cation, and therefore, the degree of swelling is exhibited low when being in contact with water.

In an exemplary embodiment of the present disclosure, bentonite uses bentonite in which interlayer ions are unsubstituted or any one selected from the group consisting of bentonite in which interlayer ions are substituted with sodium ions (Na⁺), bentonite in which interlayer ions are substituted with magnesium ions (Mg²⁺), and bentonite in which interlayer ions are substituted with potassium ions (K⁺).

Conventionally, bentonite used in a composition for treating bone diseases was used as a drug controlled-release agent, a disintegrant, an additive, and the like, and the bentonite used in the composition was calcium-based bentonite. However, the present inventors have found that bentonite itself had a therapeutic effect on bone diseases, and confirmed that in addition to calcium-based bentonite, bentonite in which interlayer ions are substituted with sodium ions (Na⁺), bentonite in which interlayer ions are substituted with magnesium ions (Mg²⁺), and bentonite in which interlayer ions are substituted with potassium ions (K⁺) also had bone disease treatment effects similar or superior to calcium-based bentonite.

In addition, compared to using bentonite as a drug controlled-release agent, a disintegrant, an additive, etc., when bentonite itself is used as an active ingredient of a therapeutic agent for bone diseases, cost may be reduced, so that economic effects may be expected.

In the present disclosure, the bentonite preferably includes 50 to 70 wt % of SiO₂, 10 to 25 wt % of Al₂O₃, 1 to 5 wt % of Fe₂O₃, and 3 to 6 wt % of MgO. In addition, the bentonite may further include at least one selected from the group consisting of 0 to 5 wt % of CaO, 0 to 5 wt % of K₂O, and 0 to 5 wt % of Na₂O.

A pharmaceutical composition according to an exemplary embodiment of the present disclosure inhibits bone resorption of osteoclasts and promotes bone formation of osteoblasts at the same time.

The composition of the present disclosure may promote the differentiation or activity of osteoblasts.

The term “osteoblasts” used herein refers to cells produced by differentiation from mesenchymal stem cells, which play a role in increasing bone density by making bony tissue, and sometimes, when the activity of osteoblasts is excessive, bone density increases to cause bone deformities or osteopetrosis.

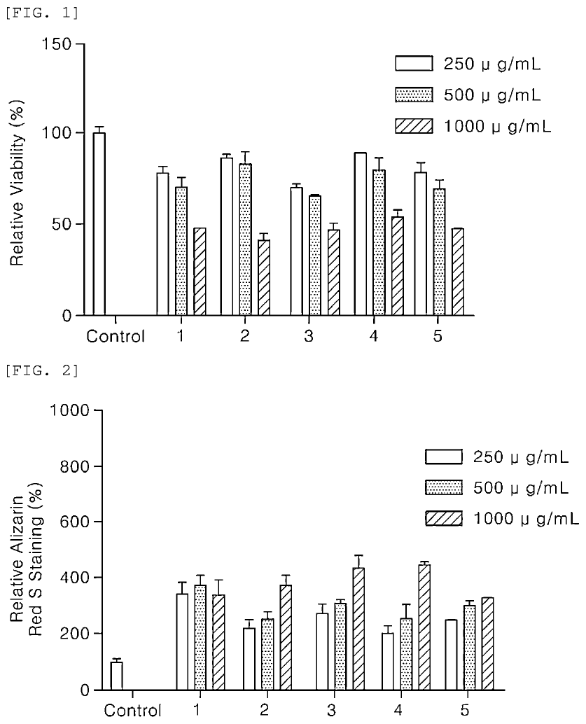

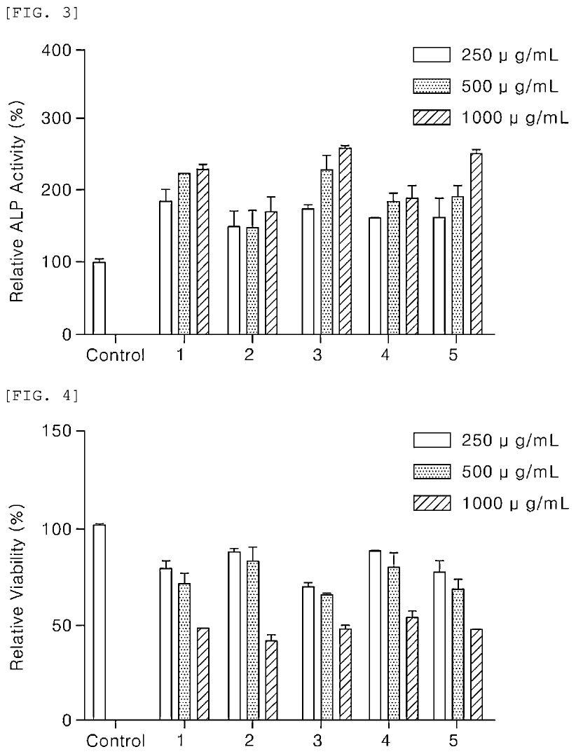

In an exemplary embodiment of the present disclosure, when osteoblasts are treated with a pharmaceutical composition containing bentonite as an active ingredient, the activity of alizarin red S as a differentiation marker was significantly increased in the initial stage of differentiation of osteoblasts (Experimental Example 1-2 and FIG. 2). In addition, in an exemplary embodiment of the present disclosure, it was confirmed that when osteoblasts are treated with a pharmaceutical composition containing bentonite as an active ingredient, the activity of alkaline phosphatase (ALP), an enzyme that was an indicator of bone formation to promotes differentiation of osteoblasts (Experimental Examples 1-3 and FIG. 3).

These results not only prove that the composition of the present disclosure may promote the differentiation of osteoblasts at the cellular level, but also suggest that the composition of the present disclosure actually increases the bone density in animals, and as a result, support that the composition of the present disclosure has effects on prevention or treatment of metabolic bone diseases such as osteoporosis by promoting the differentiation or activity of osteoblasts.

The “osteoclasts” used herein are cells derived from macrophage precursors, and osteoclast progenitors are differentiated into osteoclasts by a macrophage colony stimulating factor (M-CSF), a receptor activator NF-κB ligand (RANKL), etc., to form multinucleated osteoclasts through fusion. Osteoclasts bind to the bone through αᵥβ₃ integrin, etc. to create an acidic environment, while secrete various collagenases and proteases to cause bone resorption. Osteoclasts do not proliferate into fully differentiated cells and cause apoptosis at the end of the lifetime of about 2 weeks.

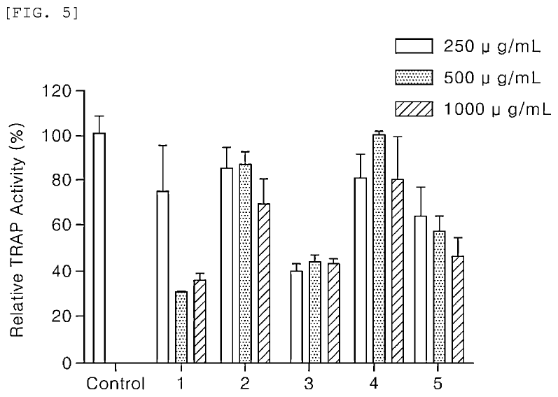

In an exemplary embodiment of the present disclosure, it was confirmed that bone marrow cells differentiated into osteoclasts were administered with a pharmaceutical composition containing bentonite as an active ingredient and then subjected to tarrateresistant acid phosphate (TRAP) as a differentiation marker for osteoclasts, and as a result, the TRAP activity was inhibited in a concentration-dependent manner (Experimental Example 2-2 and FIG. 5). As described above, since the composition containing bentonite as an active ingredient according to the present disclosure effectively inhibits the differentiation of osteoclasts through inhibition of TRAP activity, it is possible to prevent or treat metabolic bone such as osteoporosis due to increased activity of osteoclasts.

Accordingly, the composition of the present disclosure may be used for the prevention or treatment of bone metabolic diseases, and in particular, may be used for bone-related diseases caused by imbalance of osteoblasts and osteoclasts without limitations.

The pharmaceutical composition of the present disclosure may include a pharmaceutically acceptable carrier in addition to the active ingredient. At this time, the pharmaceutically acceptable carrier is those commonly used in formulation, and includes lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, etc, but is not limited thereto. Further, the pharmaceutical composition may further include a lubricant, a wetting agent, a sweetening agent, a flavoring agent, an emulsifying agent, a suspending agent, a preservative, and the like, in addition to the ingredients.

The pharmaceutical composition of the present disclosure may be administered orally or parenterally (e.g., applied intravenously, subcutaneously, intraperitoneally, or topically) according to a desired method, and a dose thereof varies depending on the condition and weight of a patient, a degree of disease, a drug form, and route and time of administration, but may be appropriately selected by those skilled in the art.

The pharmaceutical composition of the present disclosure is administered in a pharmaceutically effective dose. The ‘pharmaceutically effective dose’ used herein refers to an amount enough to treat diseases at a reasonable benefit/risk ratio applicable to medical treatment. The effective dose level may be determined according to factors including the type and severity of a disease of a patient, the activity of a drug, the sensitivity to a drug, a time of administration, a route of administration, an emission rate, duration of treatment, and simultaneously used drugs, and other factors well-known in the medical field. The pharmaceutical composition according to the present disclosure may be administered as an individual therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side-effects by considering all the factors, which may be easily determined by those skilled in the art.

As another aspect, the present disclosure provides a method for preventing or treating metabolic bone diseases, comprising administering the pharmaceutical composition to a subject.

The “subject” used herein refers to a subject in need of prevention, regulation or treatment method for diseases, and more particularly, refers to mammals such as human or non-human primates, mice, dogs, cats, horses and cattle.

Yet another object of the present disclosure relates to a health functional food for preventing or alleviating metabolic bone diseases, containing bentonite as an active ingredient.

The term “health functional food” used herein refers to a food manufactured and processed by using a specific ingredient for health supplementation as a raw material or extracting, concentrating, purifying, mixing, etc., a specific ingredient contained in a food raw material, and refers to a food designed and processed to sufficiently exhibit biological control functions such as biological defense, regulation of biological rhythm, prevention and recovery of disease, etc. by the ingredient. The composition for the health food may perform functions related to disease prevention and disease recovery.

In addition, there is no limitation in a type of health functional food in which the composition of the present disclosure may be used. In addition, the composition containing bentonite of the present disclosure as an active ingredient may be prepared by mixing known additives with other suitable auxiliary ingredients that may be contained in the health functional food according to the selection of those skilled in the art. Examples of foods to be added include meat, sausage, bread, chocolate, candy, snacks, confectionery, pizza, ramen, other noodles, gum, dairy products including ice cream, various soups, beverages, tea, drinks, alcoholic beverages, vitamin complexes and the like, and may be manufactured by adding extract, tea, jelly, juice, and the like prepared by using the extract according to the present disclosure as a main ingredient.

### EXAMPLES

Hereinafter, the present disclosure will be described in more detail with reference to Examples. However, these Examples are only illustrative the present disclosure, and the scope of the present disclosure is not limited to these Examples.

### Examples 1 to 5: Preparation of Bentonite Sample

In order to measure the activities for bone formation and bone resorption of each clay mineral, dried powder of bentonite was obtained, dissolved in 0.5% DMSO, and diluted again in a culture medium or tertiary distilled water to be used for an experiment.

As shown in Table 1 below, bentonite prepared by substituting interlayer ions of bentonite (Bgp35b-b, Bgp46b-b) and Bgp35b-b collected from a bentonite mine in Pohang and Gyeongju, Korea with Na⁺, Mg²⁺, or K⁺, respectively, was used in the experiment.

TABLE 1

| No. | Sample name | Description of sample |
|---|---|---|
| Example 1 | Bgp35b-b | Local blue |
| | | bentonite bulk |
| | | sample |
| Example 2 | Bgp35b-p-Na | Blue bentonite |
| | | purified |
| | | sample/substitution |
| | | of interlayer |
| | | ions with Na |
| Example 3 | Bgp35b-p-Mg | Blue bentonite |
| | | purified |
| | | sample/substitution |
| | | of interlayer |
| | | ions with Mg |
| Example 4 | Bgp35b-p-K | Blue bentonite |
| | | purified |
| | | sample/substitution |
| | | of interlayer |
| | | ions with K |
| Example 5 | Bgp46b-b | Local blue |
| | | bentonite bulk |
| | | sample |

### Example 2: Component Analysis of Clay Minerals

Clay minerals of Examples 1 to 5 were analyzed for components through an energy dispersive spectrometer (EDS), and the results were shown in Table 2 below.

TABLE 2

| Main | | | | | |
|---|---|---|---|---|---|
| element | Bgp35b-b | Bgp35b-p-Na | Bgp35B-p-Mg | Bgp35b-p-K | Bgp46b-b |
| SiO₂ | 54.03 | 66.08 | 53.98 | 61.01 | 60.34 |
| Al₂O₃ | 14.80 | 21.65 | 16.24 | 19.04 | 13.00 |
| Fe₂O₃ | 1.61 | 2.30 | 1.95 | 2.06 | 1.13 |
| MnO | 0.05 | 0.06 | 0.05 | 0.05 | 0.05 |
| MgO | 3.89 | 4.87 | 5.68 | 4.31 | 3.11 |
| CaO | 1.72 | ND | ND | 0.06 | 1.56 |
| Na₂O | 0.26 | 3.54 | 0.05 | ND | 0.61 |
| K₂O | ND | 0.02 | ND | 4.73 | 0.24 |
| LOI | 22.49 | 14.37 | 21.44 | 10.94 | 19.59 |
| Total | 98.85 | 112.88 | 99.38 | 102.21 | 99.63 |

### Example 3: Preparation of Cells

### Example 3-1. Preparation of Osteoblasts

MG-63 human osteoblastic cells used herein were human osteoblast-like cells (MG-63, KCLB 21427, Korean Cell Line Bank, Seoul national university college of medicine, Korea)) and cultured in an environment with a temperature of 37° C., a humidity of 100% and a 5% CO₂ concentration. The cells were cultured in a Dulbeco's modified eagle's minimum essential medium (DMEM; Hyclone, Atlanta, Ga.) containing 10% fetal bovine serum (FBS; Hyclone) and 1% antibiotics, and replaced with a new medium once every 2 days.

### Example 3-2. Preparation of Osteoclasts

RAW 264.7 cells used herein were distributed from the Korean Cell Line Bank and cultured in an environment of 37° C. temperature, 100% humidity, and 5% CO₂ concentration. The cells were cultured in a Dulbeco's modified eagle's minimum essential medium (DMEM; Hyclone, Atlanta, Ga.) containing 10% fetal bovine serum (FBS; Hyclone) and 1% antibiotics, and replaced with a new medium once every 2 days.

### Experiment Example 1: Effect of Bentonite on Promotion of Differentiation of Osteoblasts

Experimental Example 1-1. Cytotoxicity Test

The viability of the osteoblasts prepared in Example 3-1 was measured using an MTT [3-(4,5-dimethylthiazole-2-yl)-2,5-diphenyl-tetrazolium bromide] reduction method. The MTT solution confirmed the viability of cells by forming formazan by dehydrogenases of the mitochondria in living cells.

MG-63 cells were treated with the bentonite samples of Examples 1 to 5 at 250, 500, and 1000 μg/mL, respectively, and cultured at 37° C. for 24 hours at 5×10⁴ cells/well in a 96 well plate. After 24 hours, 20 μl of the MTT solution was added to each well, the cells were additionally cultured for 2 hours, the culture solution was removed, 100 μl of dimethylsulfoxide was added, and then the absorbance was measured at 570 nm, and a value of the absorbance was calculated by converting an untreated control group to 100%.

FIG. 1 is a graph comparing the results of MTT assay after treating bentonite samples to MG-63 osteoblasts according to an exemplary embodiment of the present disclosure.

Referring to FIG. 1, as a result of treating the bentonite samples of Examples 1 to 5, the cell viability was reduced to about 75%, which was determined that cytotoxicity was slightly increased.

In addition, when the bentonite samples of Examples 1 to 5 were treated at concentrations of 250 and 500 μg/mL, 75% or more cell viability was shown compared to the control group when, but when treated at a concentration of 1000 μg/mL, less than 50% of cell viability was shown, and as a result, it was shown that the cell viability for osteoblasts was varied depending on a concentration.

### Experimental Example 1-2: Mineralization Analysis

After treatment for 14 days in a 96 well plate, the degree of mineralization was measured using Alizarin Red (Sigma Chemical, St. Louis, Mo., USA) staining. The mineralization was one characteristic of ossification that occurred during osteogenesis.

MG-63 cells were treated with the bentonite samples of Examples 1 to 5 at 250, 500, and 1000 μg/mL, respectively, and immobilized with 70% (v/v) ethanol for 1 hour, and then stained with 40 mM Alizarin Red S for 1 hours in ionized water (pH 4.2) at room temperature. After sucking out and removing the Alizarin Red S solution, the stirred cells were stirred in PBS for 15 minutes on an orbital rotator. Next, the cells were washed once with fresh PBS and decolorized with 10% (w/v) cetylpyridinium chloride in 10 mM sodium phosphate (pH 7.0) for 15 minutes. The extracted strain was transferred to a 95 well plate, and the absorption capacity at 562 nm was measured using a microplate reader, and the value thereof was calculated by converting the untreated control group into 100%.

FIG. 2 is a graph of comparing results of Alizarin Red-S staining of Examples 1 to 5 according to an exemplary embodiment of the present disclosure.

Referring to FIG. 2, it was confirmed that the mineral action increased in a group treated with a previous concentration when treating bentonite according to Examples 1 to 5 in osteoblasts, MG-63, and it was confirmed that as the treated concentration was increased, the mineral action increased significantly. From these results, it was shown that bentonite enhanced the mineralization action in an MG-63 cell line, which was osteoblasts.

### Experimental Example 1-3: Measurement of ALP Activity

It was confirmed that the bentonite increased the bone density in osteoblasts by measuring ALP activity in MG-63 cells. Since the osteoblasts specifically exhibited alkaline phosphatase (ALP) activity during cell differentiation, it was confirmed whether osteoblasts were differentiated and the degree of differentiation by measuring ALP activity. By using ALP to decompose p-nitrophenylphosphate into p-nitrophenol and phosphate, an effect on osteoblasts was observed by calculating a ratio of the absorbance of each substance to the absorbance of the control group at 405 nm and measuring the ALP activity.

The cells were cultured in a 96-well plate at 5×10⁴ cells/well for 24 hours. The bentonite samples of Examples 1 to 5 were added to wells at 250, 500, and 1000 μg/mL, respectively, and continuously cultured for 3 days.

The cells were washed 3 times with physiological saline, and then cultured in a 0.1 M NaHCO₃—Na₂CO₃ buffer at pH 10 containing 0.1% Triton X-100, 1.5 mM MgCl₂ and 15 mM p-nitrophenyl phosphate at 37° C. for 1 hour, and the alkaline phosphatase activity in the cells was analyzed after an appropriate treatment period by measuring the cell activity. After stopping the reaction by adding 1 M NaOH, the absorption capacity at 405 nm was measured. The unit of phosphate activity was defined as the amount of enzyme activity at which 1 μm of p-nitrophenol per hour was isolated. The value thereof was calculated by converting the untreated control group to 100%.

FIG. 3 is a graph comparing effects on ALP activity after treating bentonite samples to MG-63 osteoblasts according to an exemplary embodiment of the present disclosure.

Referring to FIG. 3, bentonite significantly increases the activity of ALP in osteocytes, and it can be seen that such an increase in activity is concentration-dependent. From the increase in ALP activity in osteocytes, it can be seen that osteoblasts are actively differentiated. Therefore, it can be seen that bentonite is effective for bone diseases such as osteoporosis and osteopenia.

### Experiment Example 2: Confirmation of Effect of Improving Bone Differentiation of Bentonite in Osteoclasts

### Experimental Example 2-1: Cytotoxicity Test

The viability of the osteoblasts prepared in Example 3-2 was measured using an MTT [3-(4,5-dimethylthiazole-2-yl)-2,5-diphenyl-tetrazolium bromide] reduction method. The MTT solution confirmed the viability of cells by forming formazan by dehydrogenases of the mitochondria in living cells.

In order to confirm cytotoxicity, RAW 264.7 cells were cultured in a 96-well plate at 5×10⁴ cells/well at 37° C., and treated with the samples 1 to 5 of Example 1 at 250, 500, and 1000 μg/mL, respectively, and cultured for 24 hours. After 24 hours, 20 μl of the MTT solution was added to each well, the cells were additionally cultured for 2 hours, the culture solution was removed, 100 μl of dimethylsulfoxide was added, and then the absorbance was measured at 570 nm, and a value of the absorbance was calculated by converting an untreated control group to 100%.

FIG. 4 is a graph of comparing results of MTT assay after treating bentonite samples to RAW 264.7 osteoclasts according to an exemplary embodiment of the present disclosure.

Referring to FIG. 4, as a result of treating the bentonite samples of Examples 1 to 5, the cell viability was reduced to about 75%, which was determined that cytotoxicity was slightly increased.

In addition, when the bentonite samples of Examples 1 to 5 were treated at concentrations of 250 and 500 μg/mL, 75% or more cell viability was shown compared to the control group when, but when treated at a concentration of 1000 μg/mL, less than 50% of cell viability was shown, and as a result, it was shown that the cell viability for osteoblasts was varied depending on a concentration.

### Experimental Example 2-2: Experiment for Inhibiting Formation of TRAP-Positive Multinucleated Cells

The degree of TRAP proteins expressed during differentiation of osteoclasts was confirmed through staining, and the effect of bentonite on the differentiation of osteoclasts of RAW 264.7 cells was measured.

RAW 264.7 cells were placed in a 96-well plate to be 5×10⁴ cells/well, pre-incubated in a CO₂ incubator at 37° C. for 24 hours, and then treated with the bentonite samples of Examples 1 to 5 at concentrations of 250, 500, and 1000 μg/mL, respectively, together with a differentiation induction medium (50 ng/ml receptor activator of nuclear factor-KB ligand (RANKL)). The cells were incubated for a total of 5 days, the medium was removed, the cells were washed once with 500 μl PBS (pH 7.4), and treated with 3.5% formaldehyde for 10 minutes and ethanol/acetone (1:1) for 1 minute to be immobilized. Then, the cells were washed twice with 500 μl of distilled water, and then TRAP staining was performed using a leukocyte acid phosphatase kit 387-A (Sigma, USA).

The TRAP activity was obtained by treating RAW 264.7 cells with ethanol/acetone in the same manner as in the staining process, washing the cells twice with distilled water, and dispensing a 50 mM citrate buffer (pH 4.5) containing 10 mM sodium tartrate and 6 mM p-NPP (p-nitrophenylphosphate), and then reacted at 37° C. for 1 hour. After the reaction, 100 μl of an enzyme reaction solution was dispensed on a new plate, the reaction was stopped with 0.1 N NaOH, and then the absorbance was measured at 405 nm.

FIG. 5 is a graph of comparing effects on TRAP activity in RAW 264.7 cells of bentonite samples according to an exemplary embodiment of the present disclosure.

Referring to FIG. 5, it can be seen that the activity of TRAP, a differentiation marker of osteoclasts, is reduced in all of Examples 1 to 5, and it has been confirmed that bentonite inhibits osteoclasts formation and TRAP activity to inhibit bone resorption, and exhibits a positive effect on the prevention or treatment of bone diseases.

As descried above, specific examples of the pharmaceutical composition for preventing or treating metabolic bone diseases containing bentonite as an active ingredient according to an exemplary embodiment of the present disclosure have been described, but it will be apparent that various modifications are possible without departing from the scope of the invention.

Therefore, the scope of the present disclosure should not be limited to the embodiments and should be defined by the appended claims and equivalents to the appended claims.

In other words, the embodiments described above are illustrative in all aspects and should be understood as not being restrictive, and the scope of the present disclosure is represented by appended claims to be described below rather than the detailed description, and it is to be interpreted that the meaning and scope of the appended claims and all changed or modified forms derived from the equivalents thereof are included within the scope of the present disclosure.

### INDUSTRIAL AVAILABILITY

According to the present disclosure, the pharmaceutical composition or the health functional food containing bentonite as an active ingredient inhibits differentiation and bone resorption of osteoclasts and simultaneously, promotes differentiation and bone formation of osteoblasts to be usable in the prevention, alleviation or treatment of metabolic bone diseases such as osteoporosis, osteomalacia, osteopenia and bone atrophy.

In addition, according to the present disclosure, when bentonite itself is used as an active ingredient for treating bone diseases, it is possible to reduce costs compared to using bentonite as an additive and a disintegrant of a conventional composition for treating bone diseases.

1. A pharmaceutical composition for preventing or treating metabolic bone diseases, containing bentonite as an active ingredient,
wherein the bentonite includes 50 to 70 wt % of SiO₂, 10 to 25 wt % Al₂O₃, 1 to 5 wt % of Fe₂O₃, and 3 to 6 wt % of MgO.

2. The pharmaceutical composition of claim 1, wherein the bentonite is any one selected from the group consisting of bentonite in which interlayer ions are unsubstituted, bentonite in which interlayer ions are substituted with sodium ions (Na⁺), bentonite in which interlayer ions are substituted with magnesium ions (Mg²⁺), and bentonite in which interlayer ions are substituted with potassium ions (K⁺).

3. The pharmaceutical composition of claim 1, wherein the metabolic bone disease includes osteoporosis, osteomalacia, osteopenia, bone atrophy, fibrous dysplasia, Paget's disease, hypercalcemia, neoplastic destruction of bone, cancer-related bone resorption disease, osteolysis, osteoarthritis, or rheumatoid arthritis.

4. The pharmaceutical composition of claim 1, wherein the bentonite inhibits bone resorption of osteoclasts and promotes bone formation of osteoblasts at the same time.

5. (canceled)

6. The pharmaceutical composition of claim 1, wherein the bentonite further includes at least one selected from the group consisting of 0 to 5 wt % of CaO, 0 to 5 wt % of K₂O, and 0 to 5 wt % of Na₂O.

7. A health functional food for preventing or alleviating metabolic bone diseases, containing bentonite as an active ingredient,
wherein the bentonite includes 50 to 70 wt % of SiO₂, 10 to 25 wt % Al₂O₃, 1 to 5 wt % of Fe₂O₃, and 3 to 6 wt % of MgO and further includes at least one selected from the group consisting of 0 to 5 wt % CaO, 0 to 5 wt % K₂O, and 0 to 5 wt % of Na₂O.
